(19) United States
(12) Patent Application Publication
(10) Pub. No.: US 2026/0166151 A1
(43) Pub. Date: Jun. 18, 2026

(54) METHOD AND APPARATUS FOR THE ACTIVE LEARNING OF A HYBRID MODEL OF A TECHNICAL SYSTEM

(71) Applicant: Robert Bosch GmbH, Stuttgart (DE)

(72) Inventors: Katharina Ensinger, Asperg (DE); Christoph Zimmer, Stuttgart (DE); Leon Glass, Renningen (DE)

(21) Appl. No.: 19/406,463

(22) Filed: Dec. 2, 2025

(30) Foreign Application Priority Data
Dec. 11, 2024 (GE) 10 2024 211 814.1

(51) Int. Cl. A61K 45/06

(52) U.S. Cl. CPC A61K 45/06

(57) ABSTRACT

A computer-implemented method for the active learning of a hybrid model of a technical system. The hybrid model includes a physical model component and a trainable model component. The method includes: ascertaining a plurality of sequences of possible control signals for the technical system; for each of the sequences of possible control signals: ascertaining a plurality of trajectories of states of the technical system, ascertaining a first probability with which the states of the ascertained trajectories comply with a safety requirement(s), removing the sequence of possible control signals is the probability is less than or equal to a specifiable threshold value; selecting a sequence of control signals from the plurality of sequences of possible control signals; controlling the technical system with the selected sequence of control signals; measuring a resulting trajectory of states of the technical system; training the trainable model component.

### BACKGROUND INFORMATION

Zimmer et al. “Safe Active Learning for Time-Series Modeling with Gaussian Processes,” 2018, proceedings.neurips.cc/paper files/paper/2018/file/b197ffdef2ddc 3308584dce7afa3661b-Paper.pdf described the training of a time series model taking into account safety constraints.

### SUMMARY

The present invention relates to a computer-implemented method for safe active learning of a hybrid model of a technical system, in particular for selecting control signals that are both informative and safe.

Gaussian processes (GPs) with a nonlinear exogenous input structure for modeling time series are known in the prior art. In order to capture the dynamics relevant for learning the model, the input space is dynamically explored with trajectories. The input trajectories are parameterized as successive trajectory segments, which are determined step by step taking into account safety requirements and previous observations. An additional GP model is used to predict safe input regions. The segments of the input trajectory are ascertained by solving an optimization problem subject to constraints, taking into account the safety prediction. The selection of the next trajectory is based on maximizing an information gain criterion, taking into account safety constraints.

Although this approach takes safety into account in the active learning process, it has some disadvantages. Firstly, it is limited to GP models and therefore not suitable for hybrid differential equations. Secondly, it is unable to integrate prior physical knowledge.

Advantageously, the present invention proposes to use polytopic safety constraints to define the safe operating range of the technical system. This reduces the complexity of the method and ensures safety without the need for more complex sampling of a potentially high-dimensional integral over a normal distribution.

The invention allows for the selection of control signals that both maximize the acquisition of information about the system behavior and ensure compliance with safety requirements. The use of polytopic constraints also allows the application of efficient gradient-based optimization methods.

The use of explicit polytopic constraints avoids the need for a separate safety model. Furthermore, compliance with the polytopic constraints ensures the safety of the system without relying on probabilistic assumptions. The polytopic constraints also allow for the application of efficient gradient-based optimization algorithms.

In a first aspect, the invention relates to a computer-implemented method for the active learning of a hybrid model of a technical system, wherein the hybrid model comprises a physical model component and a trainable model component. According to an example embodiment, the method comprises the following steps:
ascertaining a plurality of sequences of possible control signals for the technical system;
for each of the ascertained sequences of possible control signals:
   ascertaining a plurality of trajectories of states of the technical system by means of the hybrid model given the possible sequence of control signals;
   ascertaining a first probability with which the states of the ascertained trajectories comply with a safety requirement or a plurality of safety requirements;
   removing the sequence of possible control signals from the plurality of sequences of possible control signals if the probability is less than or equal to a specifiable threshold value;
selecting a sequence of control signals from the plurality of sequences of possible control signals, wherein the sequence of control signals that has the highest value with respect to an acquisition function among the sequences of control signals of the plurality of sequences of control signals is selected;
controlling the technical system with the selected sequence of control signals;
measuring a resulting trajectory of states of the technical system;
training the trainable model component by means of the selected sequence of control signals and the measured resulting trajectory of states of the technical system.

The method can be understood in particular as the trainable model component of the hybrid model undergoing active learning through the method in the sense of machine learning, while the physical model component remains constant throughout the learning process. However, it is also possible to adjust the physical model component. By selectively choosing control signals and measuring the resulting state trajectories, new data are obtained that are used to train the trainable model component. This makes the hybrid model increasingly accurate and reliable over time.

The term “hybrid model” can be understood in particular as a mathematical model that describes the dynamics of the technical system. A hybrid model includes two different types of components: a physical model component and a trainable model component. This combination allows the use of both existing knowledge about the system and information learned from data.

The physical model component can, in particular, characterize a physical modeling of the technical system, for example based on physical laws or established engineering models. The physical model component can be understood as a priori knowledge about the system behavior and can be expressed, for example, by differential equations, algebraic equations, or other mathematical relationships.

The term “trainable model component” can be understood in particular as a “learning-capable” component of the hybrid model, in particular as a machine learning model. The trainable model component can be trained on data and can be used to compensate for the inaccuracies or incompleteness of the physical model component. It may, for example, be a neural network, a Gaussian process, or another machine learning model.

Preferably, according to an example embodiment, the hybrid model can characterize a dynamic behavior of the technical system using the formula dx/dt=f(x)+g(x), where f(x) is the physical model component, g(x) is the trainable system component and x characterizes the state of the technical system. The hybrid model can therefore, in particular, predict a change in the current state of the technical system. The hybrid model can therefore be used in the open-loop and closed-loop control of the technical system, in particular as a model of the technical system in model predictive control (MPC).

The term “state” can be understood as referring to a state of a technical system in the sense of an open-loop or closed-loop control of a dynamic system. A state can be represented in particular by a state vector. This vector can contain the values of relevant state variables of the system, for example, values that are relevant with regard to open-loop or closed-loop control of the system. The state variables can vary depending on the type of technical system, for example, position, velocity, acceleration, temperature, pressure, voltage, current and/or other physical quantities of the technical system.

The term “state” can also be understood to mean that it changes over time. The temporal evolution of the state can be determined in particular by the system dynamics of the technical system, which is represented by the hybrid model.

The term “state” can ultimately be understood as forming the basis for the safety assessment. By checking whether the state of the system meets the defined safety requirements, the safety of the system can be ensured. The safety requirements can be formulated in particular as polytopic constraints, i.e., as polytopes in the mathematical space of states, wherein the polytopes define a permissible range for the state.

The term “active learning” can be understood as the system independently “developing” new data points, which it uses to train the trainable component.

The term “safety requirement” can be understood as defining constraints on the state of the system. For example, safety requirements could specify upper limits for the temperature or pressure in a chemical reactor, minimum distances to obstacles for an autonomous vehicle, or maximum acceleration values for a robot arm.

The term “safety requirement” can be understood in particular as referring to polytopic constraints, in particular of the form Δx≤b. In this context, x is the state vector of the system, A is a matrix that linearly combines the state variables, and b is a vector that defines the upper limits for these linear combinations, i.e., the constraint. This formulation makes it possible to define complex safety limits that do not simply represent constraints on individual state variables but also take into account their interaction.

The term “sequences of possible control signals” can be understood in particular as meaning that control signals of the technical system are available for a specifiable number of time steps. The plurality of sequences of control signals then comprise these sequences of control signals. In other words, the plurality of sequences can be understood as a list of lists of control signals.

The term “ascertaining a plurality of trajectories” can be understood to mean that it includes the simulation of the hybrid model over a certain period of time for the different control signals. Starting from an initial state and a given sequence of control signals, the change of state of the system is calculated step by step by numerically solving the equations of the hybrid model.

The term “ascertaining a plurality of trajectories” can also be understood to include the ascertainment of stochastic trajectories. In the stochastic case, the model takes into account uncertainties in the model parameters or in the system behavior and provides a probability distribution over possible trajectories. In this way, a plurality of trajectories can be drawn “randomly” based on a sequence of control signals.

The term “ascertaining a plurality of trajectories” can also be understood to include the use of numerical integration methods. Various numerical methods can be used to solve the differential equations of the hybrid model, such as the Euler's method, the Runge-Kutta method or other suitable methods.

The term “acquisition function” can be understood as being able to take into account various criteria for evaluating the information gain. Examples include reducing the uncertainty of the model, maximizing mutual information, entropy, or minimizing the expected error in predicting future trajectories.

The term “acquisition function” can also be understood to mean that it takes into account the uncertainty of the predictions of the hybrid model. Usually, the greater the uncertainty of the model for a given sequence of control signals, the higher the potential information gain from conducting the corresponding experiment.

The term “acquisition function” can also be understood as being used in combination with safety requirements. In the safe active learning process, the acquisition function is not considered in isolation, but rather the sequence of control signals is selected that maximizes the acquisition function while simultaneously meeting the safety requirements. This is done by taking into account the probability, which, as described above, is calculated from the predicted trajectories and the polytopic constraints.

The term “control signal” can be understood in particular as one or more parameters that are used to influence the technical system and change its state. The control signals can be selected by the active learning algorithm in the context of the method and applied to the system to observe its response and improve the hybrid model. The control signals can take on continuous values, discrete values, or a combination of both, and depend on the specific application and the design of the technical system.

For example, in the case of a robot arm, a control signal can include the joint angles, the velocity, or the torque of the motors. In a chemical process, the control signals could be the temperature, pressure, or concentration of certain chemicals. In the context of mobile robots, such as at least partially automated vehicles, a control signal can comprise linear acceleration or velocity and/or a steering angle. In the context of safe active learning, the selection of control signals is crucial since they influence both the information acquisition and the safety of the system. The algorithm advantageously selects control signals that provide as much information as possible about the system behavior while simultaneously complying with safety requirements.

The steps of the method can be carried out iteratively in particular in order to gradually improve the hybrid model in this way. The sequences of control signals are selected and applied iteratively, with the hybrid model being updated with the newly acquired data after each iteration.

In advantageous embodiments, it is possible that the safety requirement is characterized by a polytope in the space of the states, wherein, with respect to the safety requirement, safe states lie within the polytope.

Advantageously, the polytope allows for an easily understandable statement regarding safe ranges of the state. For example, a velocity of the system can be used as part of the state, and the polytope can numerically specify a minimum and maximum velocity.

The polytope clearly indicates where the limits of the relevant state value may lie.

In advantageous embodiments, it is also possible that ascertaining the first probability comprises the following steps:
for a plurality of trajectories, preferably all trajectories, ascertaining one residual value per trajectory, wherein the residual value characterizes a minimum distance of the states of the trajectory from the limit of the polytope;
ascertaining a probability distribution of the relevant residual values;
providing a second probability as a first probability, wherein the second probability characterizes a probability with which a residual value of a trajectory is negative or zero, given the probability distribution.

In the context of this invention, the term “residual value” can be understood to mean that it quantifies the distance of a state or state trajectory from the limit of the safe operating range defined by the polytopic constraints. It is possible to calculate a residual value for each polytopic constraint and each state along a trajectory.

The term “residual value” can also be understood as being calculated using the equation r=Ax−b. Here, x is the state vector, A is the matrix of polytopic constraints and b is the vector of upper bounds. It is possible that each entry of the residual vector r represents the distance to a specific polytopic constraint.

The term “residual value” can also be understood to mean that it is negative if the corresponding state lies within the safe operating range. It is possible that a positive residual value indicates a violation of the corresponding safety constraint. The magnitude of the residual value can be a measure of the severity of the violation.

For each trajectory, a “worst” residual value can be ascertained. The worst residual value can, for example, be the largest value r with respect to all states of the trajectory. Thus, a measure for the most uncertain state of the trajectory can be used as a measure for the entire trajectory.

Regarding the plurality of trajectories, the ascertained residual values can be understood as realizations of a random variable, thus the residual value with respect to a trajectory as “drawing from a random variable.” A probability distribution can be ascertained for this random variable, in particular in the form of a probability density function; in other words, the probability distribution can be a probability density function. Preferably, a normal distribution of the residual values can be assumed and a normal distribution can be selected as the probability density function. From the residual values of the trajectories, an expected value and a standard deviation or a variance can then be estimated by means of a maximum likelihood estimation.

For the ascertained probability distribution, it is then possible to ascertain how likely it is that a residual value is positive, i.e., that at least one safety requirement is violated. For this purpose, a distribution function of the probability density function can be used in particular to ascertain the proportion of the probability density function that is negative.

In preferred embodiments, estimating the standard deviation or variance may include the following steps:
estimating an intermediate standard deviation or an intermediate variance by means of a maximum likelihood estimation;
multiplying the intermediate standard deviation or the intermediate variance by a specifiable safety value;
providing the product of the intermediate standard deviation and the safety value as the standard deviation, or providing the product of the intermediate variance and the safety value as the variance.

Advantageously, this allows the probability distribution of the residual values to be adjusted in order to incorporate a “safety buffer” or to obtain a probability of safety that can be quantified in percent. A value of 2 corresponds to a probability of approximately 2.5% in the uncertain range. A value of 3 corresponds to a probability of approximately 0.2%. The safety value is therefore a hyperparameter that can be set by the user according to their safety preferences.

Alternatively, in an example embodiment of the method above, it is also possible that, instead of a plurality of trajectories, only one trajectory is ascertained based on the sequence of possible control signals, and wherein ascertaining the probability comprises the following steps:
ascertaining a residual value with respect to the trajectory, wherein the residual value characterizes a minimum distance of the states of the trajectory from the limit of the polytope;
providing a probability of 0 if the residual value is greater than zero, and 1 otherwise.

This can be understood to mean that for each sequence of control signals, only one residual value is ascertained and only this residual value is considered. This advantageously allows the need for computing resources to be further reduced.

In preferred embodiments, it may also be provided that the trainable model component is or comprises a Gaussian process.

The hybrid model can therefore advantageously be realized through a physical model and a Gaussian process. The inventors were able to determine that a Gaussian process represents the best possible balance for the hybrid model in terms of a balance between prediction accuracy and the need for computing resources.

In advantageous embodiments, it can also be provided that the trainable model component is pre-trained by the following steps prior to the active learning steps:
receiving data, wherein the data characterize trajectories of states of the technical system;
training the trainable model component based on the received data.

Pre-training can be understood as preceding the active learning. Advantageously, the trainable model component can thus be pre-trained and better configured for the subsequent active learning. The inventors were able to determine that this could shorten the active learning process until a sufficiently high predictive accuracy of the hybrid model is achieved. In particular, this pre-training can identify an initial safe range from which the active learning method can “start.”

In the various embodiments of the invention, it may also be provided that the technical system is a mobile robot, in particular an at least partially automated vehicle, and/or that the state characterizes a position and/or a velocity and/or an acceleration of the technical system.

Embodiments of the invention are explained in detail below with reference to the figures.

### BRIEF DESCRIPTION OF THE DRAWINGS

FIG. 1 schematically shows a method for the active learning of a hybrid model according to an example embodiment.

FIG. 2 schematically shows a structure of a control system for controlling an actuator, according to an example embodiment.

FIG. 3 schematically shows an exemplary embodiment for controlling a mobile robot.

### DETAILED DESCRIPTION OF EXAMPLE EMBODIMENTS

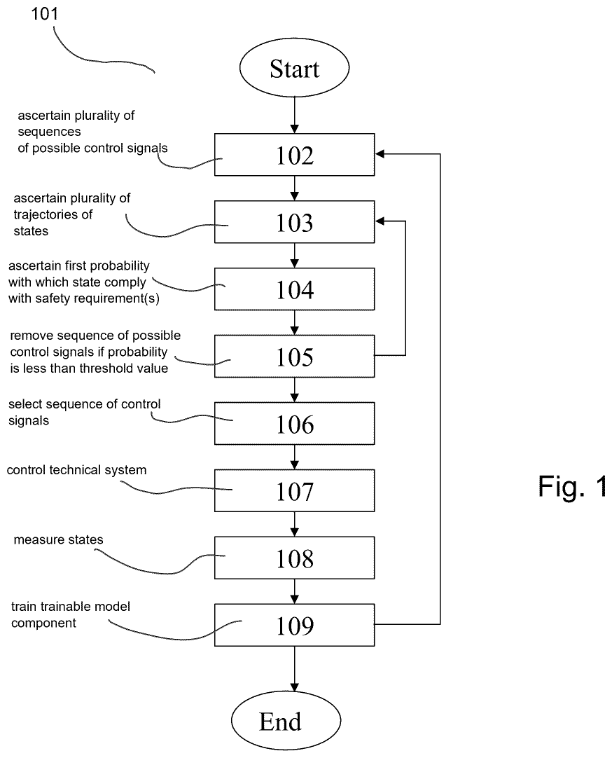

FIG. 1 schematically shows a computer-implemented method (101) for the active learning of a hybrid model of a technical system. The hybrid model comprises a physical model component and a trainable model component. The physical model component can be formulated in particular as a differential equation of the technical system. The trainable model component can be given in particular in the form of a Gaussian process. The hybrid model can combine the two model components, in particular through addition, and thus characterize the dynamic behavior of the technical system using the formula

$$\frac{d x}{d t} = f ( x ) + g ( x ) ,$$

where f(x) is the physical model component and g(x) is the trainable model component. In the exemplary embodiment, the trainable model component is preferably a Gaussian process. Preferably, the trainable model component can already be pre-trained, i.e., trained based on a training data set, prior to the steps of the method. The training steps can be carried out in particular as part of the active learning method.

In a first step (102) of the active learning method, a plurality of sequences of possible control signals for the technical system is ascertained. A control signal can be understood here as a parameter or quantity with which the technical system can be controlled, i.e., influenced. In particular, the control signals can be sequences of desired positions or actions of an actuator, e.g., a command to a brake to apply a certain brake pressure or a command to a steering system to apply a certain steering angle. In particular, the possible control signals can be randomly ascertained within a limit of allowed values.

For each of these sequences of possible control signals and starting from a current state of the technical system, a plurality of trajectories of states of the technical system is then ascertained in a second step (103) by the hybrid model. In other words, the hybrid model is used to ascertain or predict which states the technical system will assume based on the current state and the sequence of control signals executed from there. This process can in particular be probabilistic; in other words, the hybrid model can characterize uncertainty about model parameters, for example in a Gaussian process by means of the covariance function. Based on the covariance function of the Gaussian process, a plurality of trajectories can then be “drawn” based on a single sequence of control signals.

In a third step (104), a first probability is ascertained with which the states of the ascertained trajectories comply with a safety requirement or a plurality of safety requirements. A safety requirement can be defined, in particular, in the form of a polytope within which a state must be located in order to be considered safe. For example, the condition may comprise a deviation of the technical system with respect to a specified trajectory of physical positions, and a safety polytope can define a maximum deviation in the positive and negative directions.

The first probability can be ascertained in particular by considering residual values. Thus, the polytope, which imposes a safety requirement on the state, can be defined by the formula

$$A x \leq b$$

(see above). A residual value

$$r = A x - b$$

can then be understood as a numerical measure of the violation of the safety requirement: If r is negative, the safety requirement is not violated; if r is positive 0, the safety requirement is violated by the state x. The numerical value 0 can be defined in accordance with the method as positive or negative. Thus, for a trajectory of states, a residual value r can be determined at any point in time of the trajectory. In particular, a residual value can also be ascertained for different safety requirements and thus for a plurality of polytopes for each state of the trajectory.

Each trajectory can then be represented by its maximum residual value. This can be understood to mean that the residual value describes the “most dangerous” situation of the trajectory, and a trajectory is thus measured based on its smallest distance to the limit of a polytope. This value can be understood as a maximum violation of a safety requirement (if positive) or as a minimum distance for a violation of a safety requirement (negative).

For the residual values ascertained in this way for each trajectory, a probability distribution of the residual values can then be ascertained. This can be understood as meaning that the residual value over trajectories is considered a random variable, i.e., given a sequence of control signals, the actual residual value is a random variable (based on the uncertainty of the model parameters of the trainable model component). Based on the probability distribution, the probability can then be ascertained that the residual value for the sequence of control signals is positive, meaning that a safety requirement will be violated, and that the sequence of control signals is therefore unsuitable.

In particular, the residual values for each trajectory can be used to estimate an expected value and a standard deviation or variance of a normal distribution, i.e., it can be assumed that the residual values follow a normal distribution. The first probability value can then be provided as an integral of the normal distribution (given the ascertained expected value and the ascertained standard deviation or variance) from negative infinity to 0. Preferably, this can be done using a distribution function of the normal distribution.

Preferably, it is possible to multiply the standard deviation or variance by a specifiable safety value before ascertaining the integral. This creates an additional “safety buffer” by artificially increasing the variance and thus making a violation of the safety requirements more likely. The safety value can be understood as a hyperparameter of the method.

In a fourth step (105), the sequence of possible control signals is removed from the plurality of sequences of possible control signals if the probability is less than or equal to a specifiable threshold value. The probability characterizes the degree to which the corresponding sequence of control signals reliably complies with the given safety requirements. Removing sequences based on the probability can therefore be understood to mean that only sequences of control signals that can be assumed with sufficient certainty not to violate the safety requirements will actually be used in what follows.

Steps 2 to 4 are performed iteratively for all sequences of possible control signals and a plurality of safe sequences of control signals is thus ascertained.

From this plurality of safe sequences of control signals thus ascertained, a sequence of control signals is then selected in a sixth step (106), wherein the sequence of control signals that has a highest value with respect to an acquisition function among the sequences of control signals of the plurality of sequences of control signals is selected. This can be understood as first determining safe sequences of control signals and then selecting from these sequences the sequence that maximizes the acquisition function. This ensures that the sequence of control signals reliably controls the technical system and also selects a maximally informative data point for active learning purposes.

In a sixth step (107), the selected sequence of control signals is used to control the technical system according to the sequence of control signals, wherein, in a seventh step (108), the states of the technical system resulting from the control signals are measured.

In an eighth step (109), the trainable model component is trained by means of the selected sequence of control signals and the measured resulting trajectory of states of the technical system.

The steps of the method can be carried out iteratively in particular in order to gradually refine the hybrid model in this way.

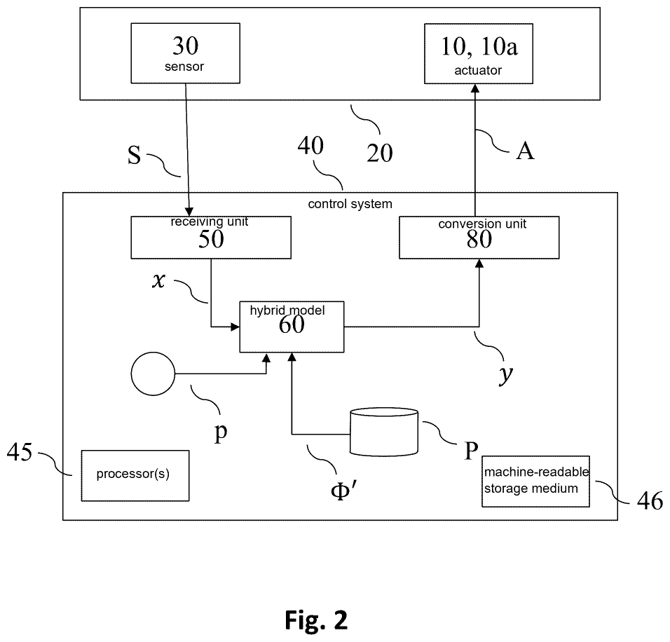

FIG. 2 shows a control system (40) which ascertains control signals (A) of an actuator (10) of a technical system by means of a hybrid model (60). At preferably regular time intervals, an environment (20) of the actuator (10) is detected using a sensor (30), in particular an imaging sensor such as a camera sensor, which can also be provided by a plurality of sensors, for example a stereo camera. The sensor signal (S)—or, in the case of a plurality of sensors, one sensor signal (S) each—of the sensor (30) is transmitted to the control system (40). The control system (40) thus receives a sequence of sensor signals (S) that characterize a state of the technical system. Therefrom, the control system (40) ascertains control signals (A) for the active learning of the hybrid model (60). The control signals (A) will be transmitted to the actuator (10).

The control system (40) receives the sequence of sensor signals (S) from the sensor (30) in an optional receiving unit (50), which converts the sequence of sensor signals (S) into a state signal (x) (alternatively, the sensor signal (S) can also be adopted directly as a state signal (x) in each case). The state signal (x) can, for example, be a portion or a further processing of the sensor signal (S). In other words, the state signal (x) is ascertained depending on the sensor signal (S). The state signal (x) represents the current state of the technical system.

The hybrid model (60) comprises a physical model component and a trainable model component (g(x)), as described in FIG. 1 and the associated exemplary embodiment. The hybrid model (60) is preferably parameterized by parameters (0) that are stored in a parameter memory (P) and are provided thereby.

From the state signal (x) and using the hybrid model (60), the control system (40) ascertains possible future state trajectories for a plurality of possible control signals (A). The outputs (y) of the hybrid model (60) characterize a trajectory of changes of a particular state using a sequence of possible control signals (p). The hybrid model (60) generates a plurality of these trajectories. This plurality is fed to a conversion unit (80), which ascertains trajectories of states of the technical system from said plurality. Based on these trajectories, the conversion unit then ascertains the first probability, as described in the exemplary embodiment of FIG. 1. Based thereon, as described in the exemplary embodiment, the sequence of control signals (A) is then ascertained, which are fed to the actuator (10) in order to control the actuator (10) accordingly.

The actuator (10) receives the control signals (A), is controlled accordingly and performs a corresponding action. Here, the actuator (10) can comprise a control logic (not necessarily structurally integrated), which ascertains, from the control signal (A), a second control signal with which the actuator (10) is then controlled. The resulting trajectory of actual states of the technical system is measured and fed to the hybrid model (60) for training the trainable model component (g(x)), as described in steps (108) and (109) of the first exemplary embodiment.

In further embodiments, the control system (40) comprises the sensor (30). In still further embodiments, the control system (40) alternatively or additionally also comprises the actuator (10).

In further preferred embodiments, the control system (40) comprises at least one processor (45) and at least one machine-readable storage medium (46) on which instructions are stored that, when executed on the at least one processor (45), cause the control system (40) to perform the method according to the invention.

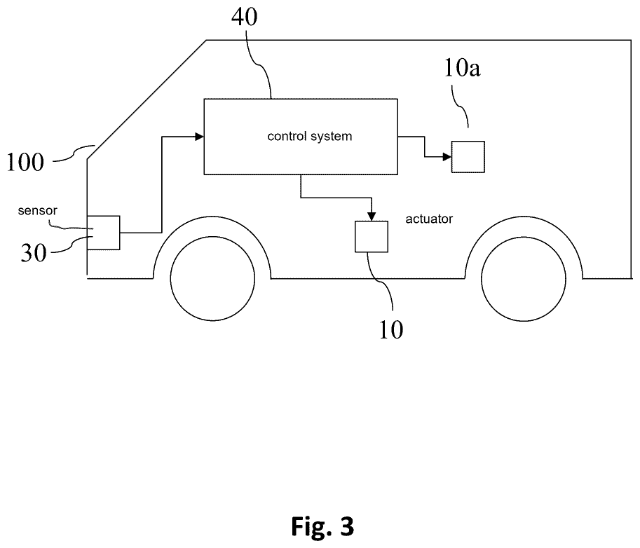

FIG. 3 shows how the control system (40) can be used to control a mobile robot, here, an at least partially automated motor vehicle (100).

The sensor (30) can, for example, be an acceleration sensor and/or velocity sensor, preferably arranged in the robot (100), of wheels or tracks of the robot (100).

The actuator (10), which is preferably arranged in the robot (100), can, for example, be a brake, a drive and/or a steering system of the motor vehicle (100).

Alternatively, the robot can also be a mobile robot other than a vehicle, for example one that moves by flying, swimming, diving or walking. The mobile robot can, for example, be an at least partially autonomous lawnmower or an at least partially autonomous cleaning robot.

The term “computer” refers to any device for processing specifiable calculation rules. These calculation rules can be in the form of software, or in the form of hardware, or even in a mixed form of software and hardware.

In general, a plurality can be understood as indexed, i.e., each element of the plurality is assigned a unique index, preferably by assigning consecutive integers to the elements contained in the plurality. Preferably, when a plurality comprises N elements, where N is the number of elements in the plurality, the elements are assigned integers from 1 to N.

1-13. (canceled)

14. A computer-implemented method for active learning of a hybrid model of a technical system, wherein the hybrid model includes a physical model component and a trainable model component, wherein the method for the active learning comprises the following steps:
ascertaining a plurality of sequences of possible control signals for the technical system;
for each of the ascertained sequences of possible control signals:
ascertaining a plurality of trajectories of states of the technical system using the hybrid model given the possible sequence of control signals,
ascertaining a first probability with which the states of the ascertained trajectories comply with a safety requirement or a plurality of safety requirements,
removing the sequence of possible control signals from the plurality of sequences of possible control signals when the probability is less than or equal to a specifiable threshold value;
selecting a sequence of control signals rom the plurality of sequences of possible control signals, wherein the sequence of control signals that has a highest value with respect to an acquisition function among the sequences of control signals of the plurality of sequences of control signals is selected;
controlling the technical system with the selected sequence of control signals;
measuring a resulting trajectory of states of the technical system which results from the controlling;
training the trainable model component using the selected sequence of control signals and the measured resulting trajectory of states of the technical system.

15. The method according to claim 14, wherein each safety requirement is characterized by a polytope in a space of the states, wherein safe states with respect to the safety requirement lie within the polytope.

16. The method according to claim 15, wherein the ascertaining of the first probability includes the following steps:
for a plurality of the ascertained trajectories, ascertaining one residual value per trajectory, wherein the residual value characterizes a minimum distance of the states of the trajectory from a limit of the polytope;
ascertaining a probability distribution of the residual values;
providing a second probability as a first probability, wherein the second probability characterizes a probability with which a residual value of the trajectory is negative or zero, given the probability distribution.

17. The method according to claim 16, wherein the probability distribution is characterized by a normal distribution, and the ascertaining of the probability function includes estimating (i) a mean value of the normal distribution, and (ii) a standard deviation or variance of the normal distribution.

18. The method according to claim 17, wherein the estimating the standard deviation or variance includes the following steps:
estimating an intermediate standard deviation or an intermediate variance using a maximum likelihood estimation;
multiplying the intermediate standard deviation or the intermediate variance by a specifiable safety value; and
providing a product of the intermediate standard deviation and the safety value as the standard deviation, or providing the product of the intermediate variance and the safety value as the variance.

19. A computer-implemented method for active learning of a hybrid model of a technical system, wherein the hybrid model includes a physical model component and a trainable model component, wherein the method for the active learning comprises the following steps:
ascertaining a plurality of sequences of possible control signals for the technical system;
for each of the ascertained sequences of possible control signals:
ascertaining a trajectory of states of the technical system using the hybrid model given the possible sequence of control signals,
ascertaining a first probability with which the states of the ascertained trajectory complies with a safety requirement, wherein the safety requirement is characterized by a polytope in a space of the states, wherein safe states with respect to the safety requirement lie within the polytope, and wherein the ascertaining of the first probability includes:
ascertaining a residual value with respect to the ascertained trajectory of states, wherein the residual value characterizes a minimum distance of the states of the trajectory from a limit of the polytope, and
providing a probability of 0 when the residual value is greater than zero, and 1 otherwise;
removing the sequence of possible control signals from the plurality of sequences of possible control signals when the probability is less than or equal to a specifiable threshold value;
selecting a sequence of control signals rom the plurality of sequences of possible control signals, wherein the sequence of control signals that has a highest value with respect to an acquisition function among the sequences of control signals of the plurality of sequences of control signals is selected;
controlling the technical system with the selected sequence of control signals;
measuring a resulting trajectory of states of the technical system which results from the controlling;
training the trainable model component using the selected sequence of control signals and the measured resulting trajectory of states of the technical system.

20. The method according to claim 19, wherein a polytopic constraint of the polytrope is characterized by the formula
$$A x \leq b ,$$
where x is a state of the technical system, A characterizes a matrix and b a numerical description of the safety requirement, and/or where the residual value is characterized by the formula
$$r = A x - b .$$

21. The method according to claim 14, wherein the trainable component is a Gaussian process.

22. The method according to claim 14, wherein the trainable model component is pre-trained by the following steps prior to the active learning steps:
receiving data, wherein the data characterize trajectories of states of the technical system;
training the trainable model component based on the received data.

23. The method according to claim 14, wherein: (i) the technical system is a mobile robot, and/or (ii) the states include a state characterizing a position and/or a velocity and/or an acceleration.

24. The method according to claim 23, wherein the robot is an at least partially automated vehicle.

25. A training apparatus configured to carry out a method for active learning of a hybrid model of a technical system, wherein the hybrid model includes a physical model component and a trainable model component, wherein the method for the active learning comprises the following steps:
ascertaining a plurality of sequences of possible control signals for the technical system;
for each of the ascertained sequences of possible control signals:
ascertaining a plurality of trajectories of states of the technical system using the hybrid model given the possible sequence of control signals,
ascertaining a first probability with which the states of the ascertained trajectories comply with a safety requirement or a plurality of safety requirements,
removing the sequence of possible control signals from the plurality of sequences of possible control signals when the probability is less than or equal to a specifiable threshold value;
selecting a sequence of control signals rom the plurality of sequences of possible control signals, wherein the sequence of control signals that has a highest value with respect to an acquisition function among the sequences of control signals of the plurality of sequences of control signals is selected;
controlling the technical system with the selected sequence of control signals;
measuring a resulting trajectory of states of the technical system which results from the controlling;
training the trainable model component using the selected sequence of control signals and the measured resulting trajectory of states of the technical system.

26. A non-transitory machine-readable storage medium on which is stored a computer program for active learning of a hybrid model of a technical system, wherein the hybrid model includes a physical model component and a trainable model component, wherein the computer program, when executed by a processor, causing the processor to perform the following steps:
ascertaining a plurality of sequences of possible control signals for the technical system;
for each of the ascertained sequences of possible control signals:
ascertaining a plurality of trajectories of states of the technical system using the hybrid model given the possible sequence of control signals,
ascertaining a first probability with which the states of the ascertained trajectories comply with a safety requirement or a plurality of safety requirements,
removing the sequence of possible control signals from the plurality of sequences of possible control signals when the probability is less than or equal to a specifiable threshold value;
selecting a sequence of control signals rom the plurality of sequences of possible control signals, wherein the sequence of control signals that has a highest value with respect to an acquisition function among the sequences of control signals of the plurality of sequences of control signals is selected;
controlling the technical system with the selected sequence of control signals;
measuring a resulting trajectory of states of the technical system which results from the controlling;
training the trainable model component using the selected sequence of control signals and the measured resulting trajectory of states of the technical system.
